(19) United States
(12) Patent Application Publication
(10) Pub. No.: US 2019/0130561 A1
(43) Pub. Date: May 2, 2019

(54) MEDICAL IMAGE PROCESSING APPARATUS

(71) Applicant: Konica Minolta Laboratory U.S.A., Inc., San Mateo, CA (US)

(72) Inventors: Shinsuke Katsuhara, Tokyo (JP); Satoshi Kasai, Tokyo (JP); Ronald Larcom, Cedar Park, TX (US)

(73) Assignee: Konica Minolta Laboratory U.S.A., Inc., San Mateo, CA (US)

(21) Appl. No.: 15/795,712

(22) Filed: Oct. 27, 2017

(51) Int. Cl. G06T 7/00; A61B 6/00

(52) U.S. Cl. CPC G06T 7/0012; A61B 6/52; G16H 30/20; G06T 2210/41; A61B 6/56; A61B 6/48

(57) ABSTRACT

A medical image processing apparatus includes a hardware processor. The hardware processor performs the following, defining a plurality of structures in an X-ray image obtained by capturing a living body; estimating signal values attributed to the structures defined in the X-ray image and generating a layer image for each of the structures; determining a factor of enhancement or attenuation for each of the structures; and enhancing or attenuating the signal value of each of the structures in the layer image based on the determined factor of enhancement or attenuation.

### BACKGROUND

### 1. Technological Field

The present invention relates to a medical image processing apparatus.

### 2. Description of the Related Art

Diagnosis of a lesion through observation of a X-ray image of the chest area is difficult because of a complicated overlapping structure of many organs, such as ribs, clavicles, blood vessels, the heart, and the diaphragm and such organs overlapping with the lesion. A single X-ray image of the chest area includes regions of different signal levels (for example, the lung field is represented in black, and low concentration areas, such as the diaphragm and the heart, are represented in white). A medical practitioner diagnoses the image by repeatedly modifying the gradation to a level suitable for diagnosis of the region. This operation is troublesome for the medical practitioner.

A bone suppression technique has been proposed to attenuate the signals corresponding to bones, such as ribs, in a X-ray image of the chest area (for example, refer to “Rib suppression in chest radiographs to improve classification of textural abnormalities”, Laurens E. Hogeweg et al., SPIE 2010). The bone suppression technology can attenuate bones in an image to enhance the visibility of lesions in the image.

Some medical practitioners, however, use ribs or other structures as anatomical landmarks for recording lesions on reports. Thus, the attenuation of all signals corresponding to bones and other structures may cause reductions in diagnostic accuracy and work efficiency. In the case of observation of the diaphragm or the posterior side of the heart, the medical practitioners should repeat the optimization of parameters such as gradation for diagnosis even if signals corresponding to bones are attenuated, and thus the diagnostic efficiency cannot be improved. Diagnosis of X-ray images of other sites also leads to the same problems if the sites to be diagnosed overlap with other structures.

### SUMMARY

An object of the present invention is to increase the diagnostic accuracy and diagnostic efficiency of X-ray images.

To achieve at least one of the abovementioned objects, according to an aspect of the present invention, a medical image processing apparatus reflecting one aspect of the present invention includes, a hardware processor: defining a plurality of structures in an X-ray image obtained by capturing a living body; estimating signal values attributed to the structures defined in the X-ray image and generating a layer image for each of the structures; determining a factor of enhancement or attenuation for each of the structures; and enhancing or attenuating the signal value of each of the structures in the layer image based on the determined factor of enhancement or attenuation.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention.

FIG. 1 illustrates the overall configuration of a X-ray image system according to an embodiment of the present invention.

FIG. 2 is a block diagram illustrating the functional configuration of the medical image processing apparatus illustrated in FIG. 1.

FIG. 3 is a flow chart illustrating a medical image display process executed by the CPU illustrated in FIG. 2.

FIG. 4 illustrates a tool for experimental calculation of signals corresponding to a structure in a medical image.

FIG. 5 illustrates an example input menu appearing on a display in step S3 in FIG. 3.

FIG. 6 is a schematic diagram illustrating the medical image display process in FIG. 3.

### DETAILED DESCRIPTION OF THE EMBODIMENTS

Details of the embodiments of the present invention will now be described with reference to the accompanying drawings. These drawings should not be construed to limit the scope of the invention.

[Configuration of X-Ray Image System 100]

The configuration will now be described.

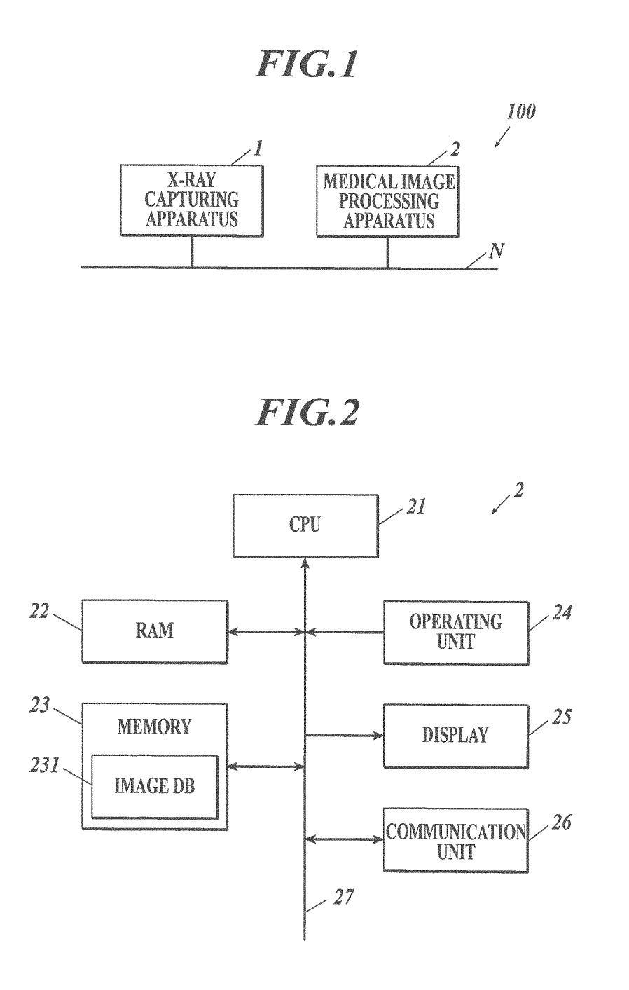

FIG. 1 illustrates the overall configuration of the X-ray image system 100 according to this embodiment. The X-ray image system 100 includes a X-ray capturing apparatus 1 and a medical image processing apparatus 2 connected to the X-ray capturing apparatus 1 via a communication network N, such as a local area network (LAN), to enable data communication between the apparatuses.

The X-ray capturing apparatus 1 includes a flat panel detector (FPD) or a computed radiographic (CR) device. The X-ray capturing apparatus 1 includes an X-ray source and an X-ray detector (FPD or CR cassette). The X-ray capturing apparatus 1 generates digital medical images (plain X-ray images) through irradiation of a target disposed between the X-ray source and the X-ray detector with X-rays and detection of X-rays transmitted through the target, and outputs the resulting images to the medical image processing apparatus 2. The medical image is outputted to the medical image processing apparatus 2 together with corresponding information, such as patient information, captured site (capturing site), and date of capturing.

The medical image processing apparatus 2 processes the medical images sent from the X-ray capturing apparatus 1 and displays the processed images for interpretation and diagnosis. With reference to FIG. 2, the medical image processing apparatus 2 includes a central processing unit (CPU) 21, a random access memory (RAM) 22, a memory 23, an operating unit 24, a display 25, and a communication unit 26 that are connected to one another via a bus 27.

The CPU 21 reads programs, such as system programs and other programs stored in the memory 23, deploys the programs in the RAM 22, and carries out various processes, such as medical image display process described below, under instructions of the deployed programs.

The RAM 22 provides a work area for temporarily storing programs read from the memory 23 and executable in the CPU 21, input or output data, and parameters during various processes the CPU 21 executes and controls.

The memory 23 includes a hard disk drive (HDD) or a non-volatile semiconductor memory. The memory 23 stores programs and data necessary for the execution of the programs, as described above, The memory 23 includes an image database (DB) 231 for storing medical images sent from the X-ray capturing apparatus 1 and layer images and combined images generated on the basis of the medical images in correlation with information, such as patient information, capturing site, and date of capturing.

The operating unit 24 includes a keyboard including cursor keys, numeral input keys, and various function keys and a pointing device, such as a mouse. The operating unit 24 sends pressed key signals in response to key operation of the keyboard or operation signals in response to mouse operation, to the CPU 21.

The display 25 includes a monitor, such as a cathode ray tube (CRT) or a liquid crystal display (LCD). The display 25 displays various menus in accordance with display signals from the CPU 21.

The communication unit 26 includes a network interface that controls data communication from/to external devices, such as the X-ray capturing apparatus 1 connected to the communication network N via a switching hub.

[Operation of X-Ray Image System 100]

The operation of the X-ray image system 100 will now be described.

The X-ray capturing apparatus 1 captures one or more images of a target. Before the image capturing, the positions of the X-ray source and the X-ray detector are adjusted such that they face each other while the subject site is positioned between the X-ray source and the X-ray detector. Then, the capturing is performed. A medical image acquired through the image capturing is sent to the medical image processing apparatus 2 via the communication network N, together with corresponding information, such as patient information, capturing site, and date of capturing.

When the communication unit 26 of the medical image processing apparatus 2 receives a medical image from the X-ray capturing apparatus 1, the CPU 21 stores the medical image in the image DB 231 in correlation with the corresponding information, such as patient information, capturing site, and date of capturing, and executes a medical image display process.

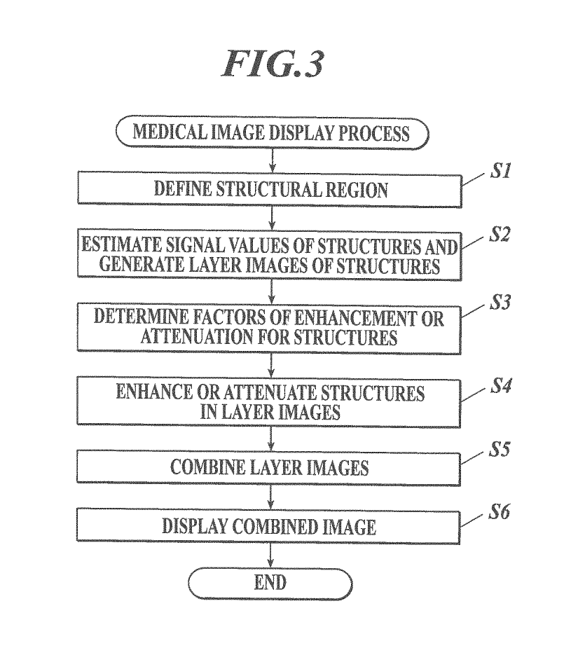

FIG. 3 is a flow chart illustrating the medical image display process executed by the CPU 21. The medical image display process is executed by the CPU 21 in cooperation with the programs stored in the memory 23. The description of this embodiment will be focused on medical images of the chest area in an anterior view.

The CPU 21 confirms a structural region in a received medical image (step S1).

Structures in a medical image of the chest area include bones, soft tissues, and medical devices. In step S1, the regions of bones and soft tissues (for example, heart, diaphragm, blood vessels, and lesions) and the regions of medical devices (for example, a pacemaker, and tubes (catheters)) are defined in the medical image. Any known method of defining such structural regions in a medical image may be employed.

A bone region can be defined through, for example, template matching of a preliminarily prepared rib template and clavicle template or a curve fitting function after edge detection, as described in U.S. Patent Application No. 2014/0079309. The defined bone region may be precisely reviewed on the basis of characteristics such as position, shape, size, concentration gradient, and direction in view of preliminary knowledge on the structure of bones, such as ribs and clavicles, to determine excessively extracted portions and remove these portions from the bone region.

A cardiac region can be defined, for example, by detecting the left and right boundary points on the outline of the heart in a medical image, fitting a model function, such as a dispersion trigonometric function, to the detected boundary points, and determining the outline of the heart on the basis of the fitted model function, as described in Japanese Patent No. 2796381.

A diaphragmatic region can be defined, for example, by capturing a medical image of the diaphragm including the lateral sides of the chest area, determining the lowest point of the diaphragm in the medical image, and defining the diaphragmatic region by the line surrounding the lowest point in the medical image (front view of the chest area) and the boundaries of the lower lung field. The lowest point can be determined, for example, by carrying out a known edge extraction process (for example, Sobel filtering or Prewitt filtering) on an image of the lateral sides of the chest area, probing an edge point from the bottom toward the top of the image, and determining the first edge point (lowest edge point) detected to be the lowest point. The boundaries of the lower lung field can be defined, for example, through selection of the edge below the lung field and protruding upward, as described in Japanese Patent Application No. 2017-510427.

A vascular region can be defined, for example, by extracting linear structures from the medical image with a Kasvand filter or a Hessian matrix, as described in Japanese Patent Application Laid-Open Publication No. 2017-18339.

A lesioned region can be defined, for example, through the technique described in Japanese Patent No. 5864542.

The region of a medical device can be defined, for example, with a classifier, such as a convolution neural network (CNN) that learns X-ray images or correct images of various medical devices, such as pacemakers and tubes, or by pattern recognition.

The CPU 21 estimates the signal values of the structural regions (signal values attributed to the structures) defined in the medical image, and generates layer images representing the signal values of the structures (step S2).

The signal value of the bones can be estimated, for example, as described in Japanese Patent Application Laid-Open Publication No. 2017-510427. That is, in an image of the lung field from which the background trend (a smooth variation in signals from the central area of the lung field to the thorax) is removed through a low-pass filter; the influence of fine signal variations (due to structures other than those corresponding to signal components of bones) is removed through morphological filtering in the extending direction of the bones; and the image is smoothened through a Gaussian filter, to estimate the signals of bones, where the direction of the morphological filtering is selected on the basis of the preliminarily obtained (known) characteristics, indicating that “signals of bones smoothly vary along the extending direction of the bones”, of images corresponding to bone signals.

Similarly, the signals of blood vessels can be estimated, for example, by removing the background trend in an image, removing the influence of fine signal variations (structures other than those corresponding to signal components of blood vessels) through morphological filtering in the extending direction of the blood vessels, and smoothing the image with a Gaussian filter. The direction of the morphological filtering is selected on the basis of the preliminarily obtained (known) characteristics, indicating that “signals of blood vessels smoothly vary along the extending direction of the bones”, of images corresponding to blood-vessel signals.

The signal values of the heart can be estimated, for example, by removing the background trend from an image, removing the influence of fine signal variations (structures other than those corresponding to signal components of the heart) through morphological filtering performed from the central area of the heart to the lateral edges of the heart, and smoothing the image with a Gaussian filter, to estimate the signals of the heart. The direction of morphological filtering is selected on the basis of the preliminarily obtained (known) characteristics, indicating that “signals of the heart smoothly vary from the central area of the heart to the lateral edges of the heart”, of images corresponding to signals of the cardiac region.

The signals of the diaphragm can be estimated, for example, by removing the background trend from an image, removing the influence of fine signal variations (structures other than those corresponding to signal components of the diaphragm) through morphological filtering performed upward from the lowest point at each horizontal position in the diaphragmatic region in the image, and smoothing the image with a Gaussian filter. The direction of morphological filtering is selected on the basis of the preliminarily obtained (known) characteristics, indicating that “signals of the diaphragm smoothly vary upward from the lowest point of the diaphragm”, of images corresponding to signals of the diaphragmatic region.

The signal values of the lesion can be estimated, for example, by extracting the frequency components in the lesion candidate region through Fourier transform and enhancing or attenuating the signals in the extracted frequency band in the lesion candidate region with a band-pass filter.

The signal values of medical devices can be estimated, for example, by a machine learning classifier, such as a deep learning classifier, on the basis of X-ray images of a chest phantom with and without medical devices placed therein.

The CPU 21 subtracts the estimated signal values of the structures from the signal values of the pixels of the original medical image, to generate a base layer image of the lung field and the torso.

Alternatively, the signal values of the structures may be estimated from energy subtraction images as correct images with a classifier, such as a CNN, that learns in pixel units.

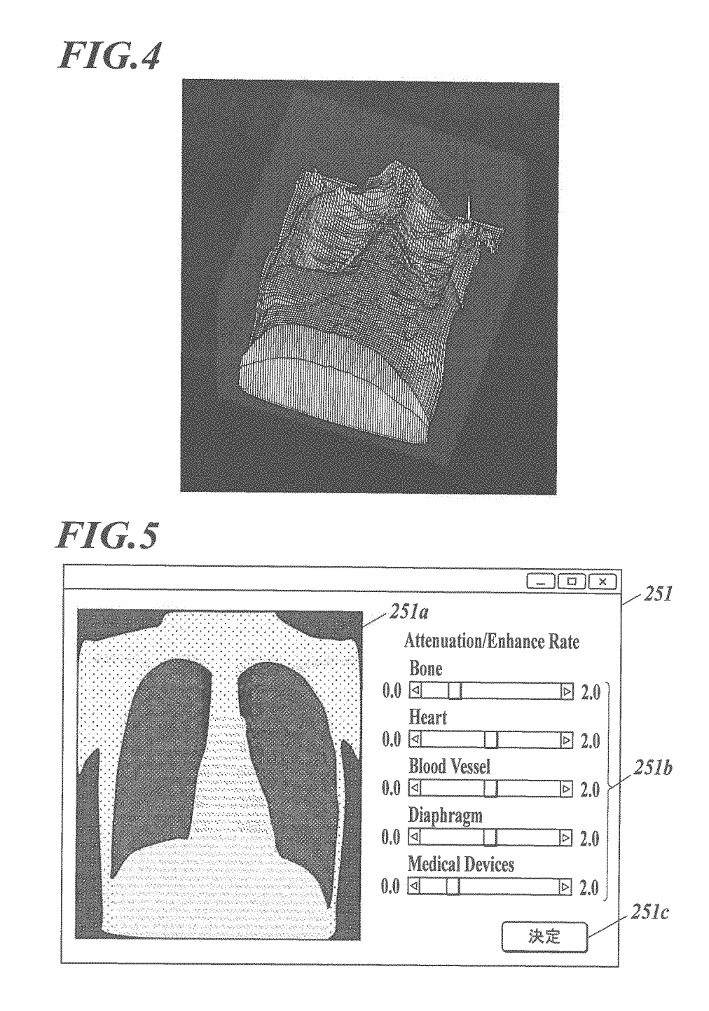

Alternatively, the signal values of the structures may be estimated with a classifier, such as a CNN, that learns correct images in pixel units, the correct image being prepared by medical practitioners and users through experimental calculation of signal values of structures defined in plain X-ray images of the chest area captured in the past in pixel units. For example, the signals of structures can be experimentally calculated through adjustment of the signal values of the pixels with a tool that represents the signal values of an image in a mesh pattern, as illustrated in FIG. 4.

The CPU 21 determines the degrees of enhancement or attenuation (enhancement/attenuation factor) of the structures (step S3).

In step S3, for example, an input menu 251 or user interface for receiving input on the enhancement/attenuation factors of the structures appear on the display 25, and the user operates the operating unit 24 on the input menu 251 to determine the enhancement/attenuation factors of the structures.

FIG. 5 illustrates an example input menu 251 appearing on the display 25 in step S3. As shown in FIG. 5, the input menu 251 includes an image display region 251a where layer images of the structures generated in step S2 appear in an overlaid manner, sliders 251b that are operated to input the enhancement/attenuation factors of the structures, and an enter button 251c that is operated to enter the enhancement/attenuation factors selected on the sliders 251b. In response to an operation of one of the sliders 251b via the operating unit 24, the CPU 21 enhances or attenuates the signal values of the layer image corresponding to the operated slider 251b, to an extent corresponding to the position of the slider 251b, and displays the resulting image in the image display region 251a. This allows the user to confirm the result of the enhancement or attenuation of the structures in the image display region 251a.

FIG. 5 illustrates the sliders 251 operated to input the enhancement/attenuation factors of the structures. Alternatively, the enhancement/attenuation factors of the structures may be input through an operation of dropdown bars or input directly in the form of numerical values.

Besides the operation by the user as described above, the enhancement/attenuation factors in step S3 may be determined, for example, on the basis of values preliminarily stored (preset factors) in the memory 23.

In this embodiment, the signals of the lung field are not a target of enhancement or attenuation. Alternatively, the signals of the lung field may be a target of enhancement or attenuation.

Alternatively, the enhancement/attenuation factors of the structures including different injuries and diseases, such as lung cancer, possible bone fractures, and pneumoconiosis, may be preliminarily stored (preset factors) in the memory 23 in correlation with corresponding injure or disease names, so that the CPU 21 can retrieve the enhancement/attenuation factors of the structures corresponding to a specific injury or disease selected via the operating unit 24 from the memory 23 and determine the retrieved values as the enhancement/attenuation factors of the structures.

Alternatively, the enhancement/attenuation factors of the structures preliminarily selected by different users may be preliminarily stored (preset factors) in the memory 23 in correlation with the corresponding user IDs, and the CPU 21 may retrieve the enhancement/attenuation factors of the structures corresponding to the user ID of the logged in user from the memory 23 and determine the retrieved value as the enhancement/attenuation factors of the structures.

Alternatively, the enhancement/attenuation factors of the structures depending on the medical facility may be preliminarily stored (preset factors) in the memory 23, and the CPU 21 may retrieve the values of the enhancement/attenuation factors of the structures from the memory 23 and determine the enhancement/attenuation factors of the structures as the retrieved values.

Alternatively, the enhancement/attenuation factors of the structures for different clinical departments, such as the respiratory division and the orthopedic division, may be preliminarily stored (preset factors) in correlation with corresponding clinical department names in the memory 23, and the CPU 21 may retrieve the values of the enhancement/attenuation factors of the structures corresponding to the clinical department name selected by the operating unit 24 from the memory 23 and determine the enhancement/attenuation factors of the structures as the retrieved values.

Alternatively, the CPU 21 may accumulate input history of the enhancement/attenuation factors of the structures input by users in the memory 23 and determine the enhancement/attenuation factors of the structures on the basis of the input history. For example, representative values, such as the average, the median, the maximum, or the minimum, of the enhancement/attenuation factors of the structures in the input history may be calculated and determined as the representative values of the enhancement/attenuation factors of the structures.

Alternatively, the CPU 21 may cause layer images having signal values enhanced or attenuated in accordance with the preliminarily stored enhancement/attenuation factors of the structures to appear on the display 25 in an overlaid manner, cause a user interface, such as slider bars, for adjustment of the enhancement/attenuation factors of the structures to appear, and adjust the enhancement/attenuation factors of the structures in accordance with the input via the user interface.

If more than one set of enhancement/attenuation factors of the structures is preliminarily stored in the memory 23, it is preferred that the user preliminarily select the set to be used through the operation of the operating unit 24.

The CPU 21 enhances or attenuates the signal values of the pixels of the structural regions in the layer images in accordance with the enhancement/attenuation factors of the corresponding structures determined in step S3 (step S4).

For example, if the enhancement factor is α, the signal value after enhancement is α×signal value. If the attenuation factor is β, the signal value after attenuation is β×signal value. At the maximum attenuation, the factor β equals zero.

The CPU 21 combines the enhanced or attenuated layer images (step S5) and causes the combined image to appear on the display 25 (step S6). The CPU 21 then ends the medical image display process.

The signal values of the pixels in the layer images are added to generate a combined image.

The layer images and the combined image are stored in the image DB 231 of the memory 23 in correlation with the original medical image.

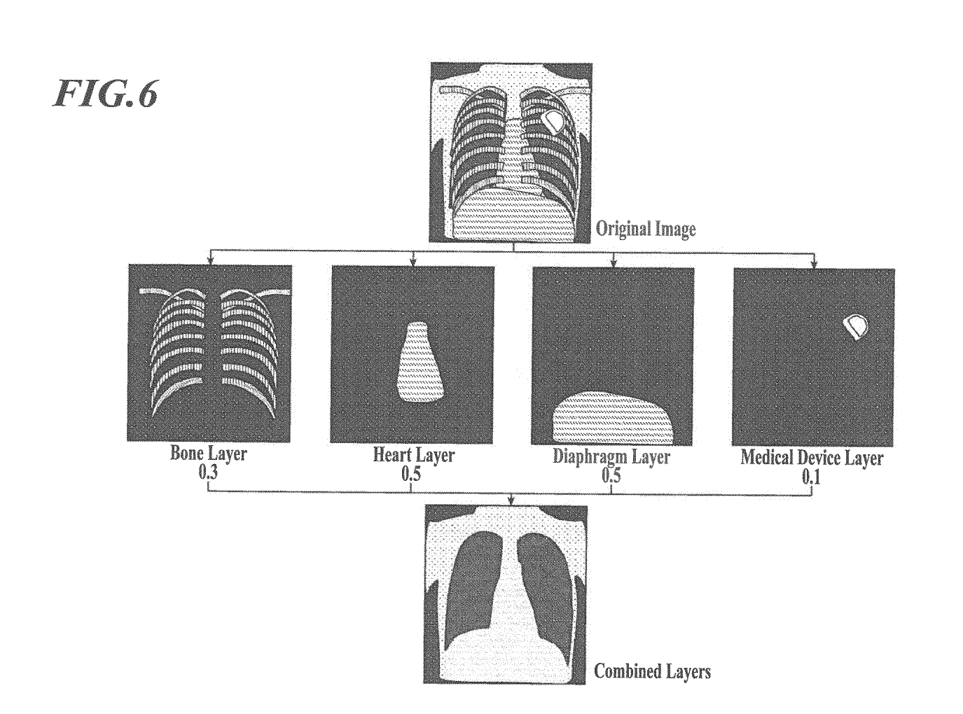

FIG. 6 is a schematic view of the processing involving the medical image display process.

With reference to FIG. 6, the medical image display process involves estimation of the signal values of the structures in a medical image (original image) to generate layer images of the structures, determination of the enhancement/attenuation factors of the structures, and enhancement or attenuation of the structural regions of the layer images with the determined enhancement/attenuation factors. The enhanced or attenuated layer images are overlaid and combined.

This allows the enhancement/attenuation factor of the signal to be determined for each structure. Thus, the structures in the medical image can be enhanced or attenuated in accordance with the objective of the clinical treatment and preference by the user. This leads to increases in diagnostic accuracy and diagnostic efficiency.

The X-ray image system and the medical image processing apparatus according to the present invention should not be limited to those according to the embodiments described above.

For example, in the medical image display process described above, the CPU 21 causes a combined image including enhanced or attenuated structures to appear on the display 25. Alternatively, layer images may appear on the display 25 in an array. In this way, the user can observe the individual structures.

Alternatively, the signal values of the layer image of one or more structures of the overlaid layer images may be automatically enhanced or attenuated by a predetermined enhancement/attenuation factor, and the resulting image may be displayed on the display 25. This enables the user to observe the image including the structures having varied enhancement/attenuation factors without shift of the line of sight.

In the medical image display process described above, the medical image is a plain X-ray image of the chest area. Alternatively, the medical image may be a plain X-ray image of any other area, such as the abdomen or the head, at which structures overlap with each other. In the medical image display process described above, the medical image is a single plain X-ray image. Alternatively, the medical image may be an X-ray moving image including consecutive plain X-ray images captured at predetermined time intervals, such as a dynamic image of a target in motion. In such a case, steps S1 to S5 may be carried out on each frame image of the X-ray moving image.

In the embodiment described above, the structures to be enhanced or attenuated include bones, blood vessels, the heart, the diaphragm, lesions, and medical devices. Alternatively, one or more of these structures may be enhanced or attenuated. Moreover, the lung field may be included in the structures to be enhanced or attenuated. Alternatively, the structures to be enhanced or attenuated that are to be captured in layer images may be selected by a user operation of the operating unit 24.

In the description above, a HDD or a non-volatile semiconductor memory serves as a computer readable medium storing the program according to the present invention. Any other computer readable medium is also available. Alternatively, the computer readable medium may be a portable recording device, such as a CD-ROM. Carrier waves may also be applied to the present invention as a medium that provides data of the program according to the present invention via a communication line.

The detailed configuration and operation of the components of the X-ray image system 100 according to the embodiments described above may be appropriately modified without departing from the scope of the present invention.

The embodiments described above should not be construed to limit the present invention, and the claims, other equivalents thereof, and modifications thereof are included in the scope of the invention.

What is claimed is:

1. A medical image processing apparatus comprising:
a hardware processor:
defining a plurality of structures in an X-ray image obtained by capturing a living body;
estimating signal values attributed to the structures defined in the X-ray image and generating a layer image for each of the structures;
determining a factor of enhancement or attenuation for each of the structures; and
enhancing or attenuating the signal value of each of the structures in the layer image based on the determined factor of enhancement or attenuation.

2. The medical image processing apparatus according to claim 1, wherein the hardware processor further combines a plurality of the layer images to generate a combined image.

3. The medical image processing apparatus according to claim 1, wherein the hardware processor further estimates the signal values attributed to the structures through smoothing of regions corresponding to the structures in the X-ray image, based on preliminarily obtained characteristics of the structures.

4. The medical image processing apparatus according to claim 1, wherein the hardware processor determines the factor of the enhancement or attenuation of each structure to be preset factors of enhancement or attenuation.

5. The medical image processing apparatus according to claim 1, wherein the hardware processor determines the factors of the enhancement or attenuation of the structures based on an input through a user interface.

6. The medical image processing apparatus according to claim 1, wherein the hardware processor determines the factor of the enhancement or attenuation of each structure based on an input history of the factor of the enhancement or attenuation of the structure through a user interface.
